(19) United States
(12) Patent Application Publication
(10) Pub. No.: US 2022/0265964 A1
(43) Pub. Date: Aug. 25, 2022

(54) BENT TUBE UNIT OF MEDICAL GUIDE SHEATH

(71) Applicant: OLYMPUS CORPORATION, Tokyo (JP)

(72) Inventors: Keiichi ASAMI, Tokyo (JP); Tomonori IMAMURA, Tokyo (JP); Kinji TAKIGUCHI, Tokyo (JP)

(73) Assignee: OLYMPUS CORPORATION, Tokyo (JP)

(21) Appl. No.: 17/742,700

(22) Filed: May 12, 2022

Related U.S. Application Data
(63) Continuation of application No. PCT/JP2019/047643, filed on Dec. 5, 2019.

(51) Int. Cl. A61M 25/01; A61B 1/005

(52) U.S. Cl. CPC A61M 25/01; A61B 1/0051

(57) ABSTRACT

Provided is a bendable tube unit of a medical guide sheath. The bendable tube unit includes, a coil in which an elastic wire is wound in a spiral form, and a rod that is arranged inside the coil along and n near a generating line in a circumferential direction of the coil, and that has a distal end portion and a rear end portion, the distal end portion being fixed to a front end portion of the coil, the rear end portion being connected to an operating portion configured to apply a bending action to the coil, the rod being configured to bend the coil by moving toward a distal end side of the coil to apply a stretching force to widen a space between parts of the wire near the generating line deviated from an axis of the coil.

### CROSS-REFERENCE TO RELATED APPLICATION

This application is a continuation of International Application No. PCT/JP2019/047643, filed on Dec. 5, 2019, the entire contents of which are incorporated herein by reference.

### BACKGROUND

### 1. Technical Field

The present disclosure relates to a bendable tube unit of a medical guide sheath.

### 2. Related Art

Laparoscopic surgery is a procedure in which holes of several millimeters (mm) to several tens of mm are made in the flesh in the abdomen at multiple sites, and an endoscope or a therapy instrument are inserted through the holes to perform resection of a lesion on an outer wall of an organ, or partial or complete removal of an organ are performed, unlike a procedure of inserting an endoscope from a mouth of a subject to resect a lesion on an inner wall of a digestive tract. The laparoscopic surgery is a minimally invasive procedure in which a burden on a subject is smaller than laparotomy. In the laparoscopic surgery, when insertion and removal of an endoscope or a therapy instrument occur frequently, a procedure is performed in a state in which a passage to an affected area is prepared by using a tool, such as a guide sheath, to shorten procedure time, and to obtain the observation field of view of the endoscope.

For example, in Japanese Patent No. 3539124, a guide sheath having an insertion portion that is bendable in a predetermined direction by pivotably attaching plural angle rings sequentially at a distal end of a rigid pipe is described.

In Japanese Unity Model Registration No. 2524905, a bendable tube that includes a fixing means to hold a spiral tube in a state in which it is curved toward one side, and a bending means that applies a force to the spiral tube in a direction opposite to a curved direction by the fixing means is described.

### SUMMARY

In some embodiments, provided is a bendable tube unit of a medical guide sheath. The bendable tube unit includes, a coil in which an elastic wire is wound in a spiral form, and a rod that is arranged inside the coil along and near a generating line of a circumferential direction of the coil, and that has a distal end portion and a rear end portion, the distal end portion being fixed to a front end portion of the coil, the rear end portion being connected to an operating portion configured to apply a bending action to the coil, the rod being configured to bend the coil by moving toward a distal end side of the coil to apply a stretching force to widen a space between parts of the wire near the generating line deviated from an axis of the coil.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a plane view illustrating a configuration of a guide sheath according to a first embodiment of the disclosure;

FIG. 2 is a cross-section illustrating a configuration of the guide sheath according to the first embodiment of the disclosure;

FIG. 3 is a cross-section illustrating a configuration of a principal part of the guide sheath according to the first embodiment of the disclosure;

FIG. 4 is a cross-section explaining about an insertion portion in a state in which a bendable portion is bent;

FIG. 5 is a cross-section illustrating a configuration of a principal part of a guide sheath according to a first modification of the first embodiment of the disclosure;

FIG. 6 is a cross-section for explaining a configuration of a principal part of a coil of an insertion portion in the guide sheath according to a second modification of the first embodiment of the disclosure;

FIG. 7 is a cross-section for explaining a configuration of a principal part of a coil of an insertion portion in the guide sheath according to a third modification of the first embodiment of the disclosure;

FIG. 8 is a partial cross-section illustrating a configuration of a principal part of a guide sheath according to a second modification of the disclosure;

FIG. 9 is a cross-section for explaining about an insertion portion in a state in which a bendable portion is bent;

FIG. 10 is a cross-section in which a part of a coil illustrate in FIG. 9 is enlarged;

FIG. 11 is a cross-section for explaining a configuration of a principal part of a coil in a state in which the insertion portion is bent in the guide sheath according to the third embodiment of the disclosure;

FIG. 12 is a diagram explaining about the coil of the insertion portion in a state in which the bendable portion is bent;

FIG. 13A is a diagram (part 1) explaining a manufacturing method of the coil in the guide sheath according to the third embodiment of the disclosure;

FIG. 13B is a diagram (part 2) explaining the manufacturing method of the coil in the guide sheath according to the third embodiment of the disclosure;

FIG. 13C is a diagram (part 3) explaining the manufacturing method of the coil in the guide sheath according to the third embodiment of the disclosure;

FIG. 13D is a diagram (part 4) explaining the manufacturing method of the coil in the guide sheath according to the third embodiment of the disclosure;

FIG. 14 is a cross-section for explaining a configuration of a principal part of a coil of an insertion portion in a guide sheath according to a first modification of the third embodiment of the disclosure;

FIG. 15 is a cross-section for explaining a configuration of an insertion portion in a guide sheath according to a second modification of the third embodiment of the disclosure;

FIG. 16 is a diagram explaining a configuration of a coil in a guide sheath according to a fourth embodiment of the disclosure; and

FIG. 17 is a diagram explaining a configuration of an insertion portion in a guide sheath according to a fifth embodiment of the disclosure.

### DETAILED DESCRIPTION

Hereinafter, forms (hereinafter, “embodiments”) to implement the disclosure will be explained in detail with reference to the accompanying drawings. Note that the drawings are schematic illustration, and dimensional relationships and ratios differ from an actual situation.

Moreover, also among the drawings, parts in which dimensional relationships and ratios differ from one another are included.

### First Embodiment

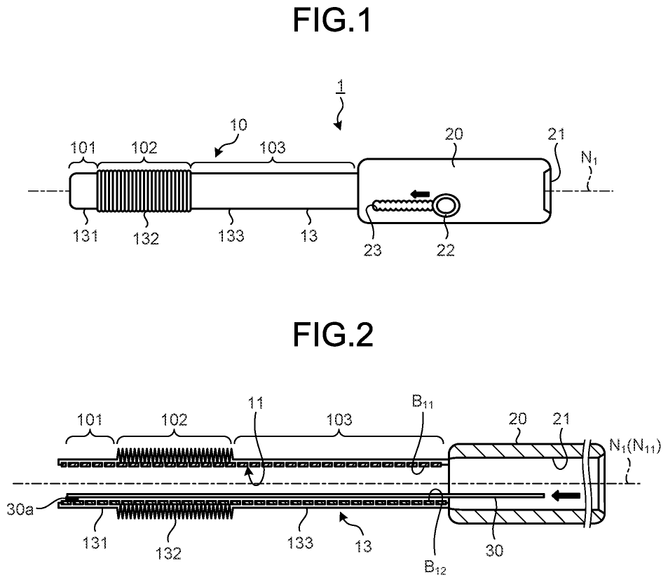

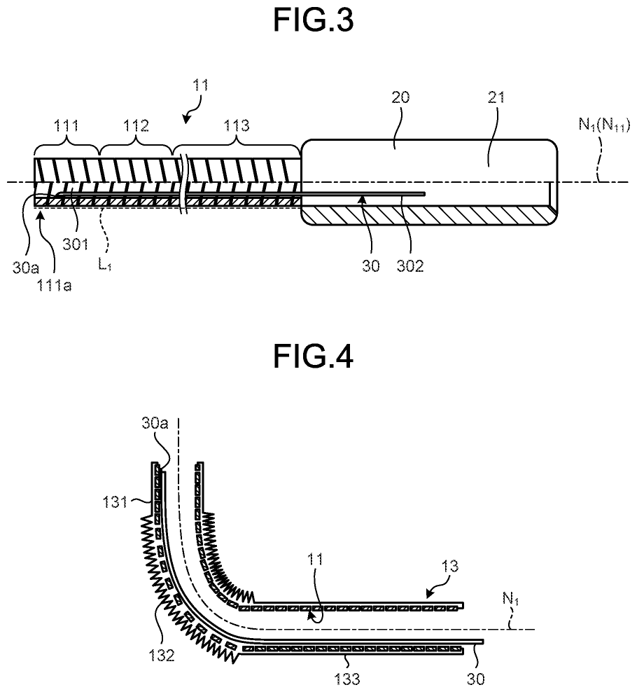

FIG. 1 is a plane view illustrating a configuration of a guide sheath according to a first embodiment of the disclosure. FIG. 2 is a cross-section illustrating a configuration of the guide sheath according to the first embodiment of the disclosure. FIG. 3 is a partial cross-section illustrating a configuration of a principal part of the guide sheath according to the first embodiment of the disclosure. FIG. 3 is a partial cross-section (half section) of an operating portion 20 cut on a plane parallel to a longitudinal axis N₁ of guide sheath 1 illustrated in FIG. 1 and including the longitudinal axis N₁. FIG. 3 illustrates an insertion portion from which a cover member 13 is removed in the guide sheath 1 illustrated in FIG. 1. In FIG. 2 and the like in a cross-section of a coil, an outline of a wire positioned behind a cross-sectional plane are omitted.

The guide sheath 1 extends in a cylindrical shape, and a part thereof is inserted in a hole formed on a body surface of the subject. Thereafter, an endoscope or a therapy instrument are inserted to the guide sheath 1 to perform observation or treatment inside the subject.

The guide sheath 1 includes an insertion portion 10 and an operating portion 20. The insertion portion has a cylindrical shape and is configured to be inserted into the subject. The operating portion 20 has a cylindrical shape, is connected to a proximal end side of the insertion portion 10, and is configured to operate a bent mode of the insertion portion 10. In the insertion portion 10 and the operating portion 20, through holes communicating with each other are formed. In the present embodiment, a center axis of the through hole of the insertion portion that is not bent and a center axis of the through hole of the operating portion 20 coincide respectively with the longitudinal axis N₁ of the guide sheath 1. Hereinafter, in the insertion portion 10, a side closer to the operating portion 20 in a longitudinal direction (direction of the longitudinal axis N₁) is denoted as a proximal end (rear end) side, and the opposite side is denoted as a distal end (front end) side.

The insertion portion 10 includes a distal end portion 101 arranged on the distal end side, a bendable portion 102 that extends from the distal end portion 101 toward the proximal end side and that can be bent in a predetermined one direction, and a proximal end portion 103 that extends from the bendable portion 102 toward the side opposite to the distal end portion 101 (toward the side of the operating portion 20). The insertion portion 10 includes a coil 11 as its internal structure, and the coil 11 is made up of a wire that extends with a sectional plane maintained in a round-cornered rectangular shape and that is wound in a spiral form. The coil 11 has a uniform wound angle, or a wound angle within a predetermined range. The wire constituting the coil 11 has elasticity.

In the operating portion 20, an opening portion 21 in which an endoscope or a therapy instrument is inserted is formed. The operating portion 20 includes an operating lever 22 to bend the bendable portion 102 in a predetermined direction. Moreover, in the operating portion 20, a control groove 23 that extends in the direction of the longitudinal axis N₁ of the guide sheath 1, and that controls a moving range of the operating lever 22 is formed. In the control groove 23, a side surface extending in the direction of the longitudinal axis N₁ has a wavy form, and has a form in which a portion with an opening having a large width (first portion) and a portion with an opening having a small width (second portion) are repeatedly arranged in the direction of the longitudinal axis N₁. The operating lever 22 moves the guide sheath 1 in the longitudinal axis N₁ along the control groove 23. At this time, because a side surface of the control groove 23 has waves along the direction of the longitudinal axis N₁ and has a structure of engaging the operating lever 22 in the respective second portions while the operating lever 22 moves, it is possible to change a position of the operating lever 22 in the control groove 23 stepwise, and to stop at the position of the operating lever 22. At the stopped position, by turning the operating lever 22, the operating lever 22 is screwed to a main body of the operating portion 20 to be fixed. The operating portion 20 is grabbed by an operator, such as a doctor, at its use.

In the guide sheath 1, an operating rod 30, one end (distal end portion 301) of which is fixed to an inner periphery (surface on an inner side of the wire of the coil 11 in this example) of a front end portion 111a of the coil 11, and the other end (rear end portion 302) of which is connected to the operating lever 22 is arranged. The operating rod 30 has a belt shape that is elongated to have a uniform rectangular section. The operating rod 30 extends along a generating line L₁ in a peripheral direction of the coil 11. The generating line herein signifies a straight line substantially parallel to a direction of a spiral axis N₁₁ of the coil 11 on a cylindrical surface formed with an outer diameter curved surface of the coil 11. The operating rod 30 moves in the direction of the longitudinal axis N₁ following movement of the operating lever 22. The operating rod 30 is fixed to a distal end of the coil 11 by a fixing portion 30a. The fixing portion 30a is formed by, for example, laser welding, brazing or solder, adhesion, resistance welding, or the like. The operating rod 30 is fixed to the operating lever 22 by a publicly known fastening means, such as screwing. The operating rod 30 corresponds to a forward and backward movement transmitter, and is formed by using a material having flexibility and snapping property, such as stainless steel. The coil 11 and the operating rod 30 form a bendable tube unit.

The coil 11 is formed by winding the wire around the spiral axis N₁₁. Specifically, the coil 11 has a close-wound structure in which adjacent parts of the wire in the direction of the spiral axis N₁₁ contact each other. The coil 11 includes a first rigid coil portion 111 that extends corresponding to an area in which the distal end portion 101 is formed, a bendable coil portion 112 that extends corresponding to an area in which the bendable portion 102 is formed, and a second rigid coil portion 113 that extends corresponding to an area in which the proximal end portion 103 is formed (refer to FIG. 3). Hereinafter, in the coil 11, a distal end side in the direction of the spiral axis N₁₁ corresponds to one end side, and a proximal end side in the direction of the spiral axis N₁₁ corresponds to the other end side. In the first embodiment, the spiral axis N₁₁ coincides with the longitudinal axis N₁ in the guide sheath 1 assembled with the coil 11.

Returning back to FIG. 1, 2, an outer periphery of the coil 11 is covered with the cover member 13. The cover member 13 suppresses damaging an organ or the like in the subject with projections and depressions of the coil 11. The cover member 13 includes a first cover portion 131 that is formed at a position corresponding to the distal end portion 101, a stretching portion 132 in a bellows shape formed at a position corresponding to the bendable portion 102, and a second cover portion 133 that is formed at a position corresponding to the proximal end portion 103. The cover member 13 is formed by using a stretchable material, such as a rubber material.

The operating rod 30 is set to have such a width and a thickness enabling to prevent the operating rod 30 from buckling by depression of the operating lever 22 based on a capacity (spring constant) of the coil 11, and a power necessary for the stretching portion 132 of the cover member 13 to stretch and shrink. The operating rod 30 is bent following a bent of the bendable coil portion 112.

FIG. 4 is a cross-section for explaining about the insertion portion 10 in a state in which the bendable portion 102 is bent. When the operating rod 30 is pushed toward the insertion portion 10 (the distal end side) by movement of the operating lever 22, the coil 11 is pulled by the first rigid coil portion 111 and is pressed to be expanded. At this time, a space between adjacent parts of the wire in the direction of the spiral axis N₁₁ widens according to the pulling force of the operating rod 30, and the coil 11 is deformed. Moreover, as the bendable coil portion 112 is bent, the stretching portion 132 stretches. At this time, by engaging the operating lever 22 in the control groove 23, the bent state of the bendable portion 102 can be maintained.

A load on the operating rod 30 in pushing can be adjusted by changing the thickness or the width, and a quality of material (elasticity, and the like) of the operating rod 30 in a belt shape extending with a rectangular section maintained.

Because the first embodiment explained above enables to form a bent state with the coil 11 that is constituted of a single member formed by winding the wire, unlike a configuration in which an insertion portion is bent by plural angle rings, it is possible to assemble a bendable tube (coil 11 in this example) easily, and to reduce time and effort of an assembly work because the bendable tube can be handled as a unit. Moreover, according to the first embodiment, because a bending operation is enabled by the operating rod 30 in a belt shape extending with the rectangular section maintained, it is possible to suppress deformation of the operating rod 30 in a width direction (direction perpendicular to a plane passing through the spiral axis N₁₁ in this example), and to suppress shaking in a vertical direction relative to a direction of bent.

Furthermore, in the first embodiment, because a bendable mechanism is formed by using the coil 11 constituted of wound the wire, it is possible to reduce the number of parts and to simplify the structure compared to the configuration of bending the insertion portion by plural angle rings, and to make assembly of the insertion portion easy.

First Modification of First Embodiment

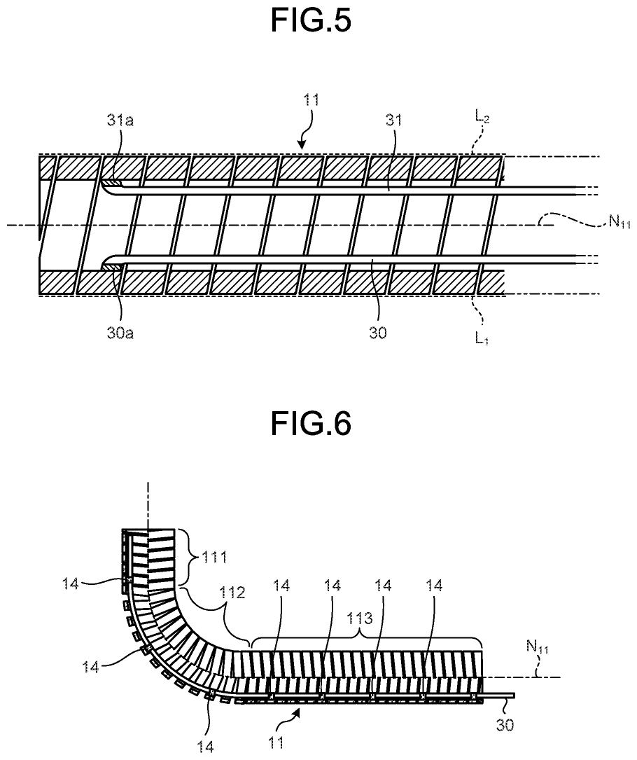

FIG. 5 is a cross-section for explaining a configuration of a principal part of a coil of an insertion portion in a guide sheath according to a first modification of the first embodiment of the disclosure. The first modification differs from the first embodiment described above in the number of arranged operating rods. Configuration other than this point is the same as that of the first embodiment. Hereinafter, a configuration different from the first embodiment described above will be explained.

In the guide sheath according to the first modification, in addition to the operating rod 30 described above, an operating rod 31, one end of which is fixed to an inner periphery of the coil 11, and the other end of which is connected to operating lever 22 is arranged. The operating rod has a belt shape extending with a rectangular section maintained. The operating rod 31 extends along a generating line L₂ in a circumferential direction of the coil 11. The generating line L₂ is a generating line across the spiral axis N₁₁ from the generating line L₁. The operating rods 30, 31 are arranged along and near respective generating lines at equal angles in the circumferential direction of the coil 11 about the spiral axis N₁₁. The operating rod 31 moves in the direction of the longitudinal axis N₁ following movement of the operating lever 22, similarly to the operating rod 30. A fixing portion 31a is by, for example, laser welding, brazing or solder, adhesion, resistance welding, or the like. The operating rod 31 is fixed to the operating lever 22 by a publicly known fastening means, such as screwing. The operating rod 31 is formed by using a material having flexibility and snapping property, such as stainless steel. The coil 11 and the operating rods 30, 31 form a bendable tube unit.

According to the first modification explained above, even when two operating rods are provided, similarly to the first embodiment described above, it is possible to assemble a bendable tube (the coil 11 in this example), and to reduce time and effort in an assembly work because the bendable tube can be handled as a unit. Moreover, according to the first modification, because a bending operation is enabled by the operating rod 30 in a belt shape extending with the rectangular section maintained, it is possible to suppress deformation of the operating rod 30 in a width direction (direction perpendicular to a plane passing through the spiral axis N₁₁ in this example), and to suppress shaking in a vertical direction relative to a direction of bent. At this time, the bending operation is enabled to bend in two directions in addition to the bending operation of the first embodiment.

Second Modification of First Embodiment

FIG. 6 is a cross-section for explaining a configuration of a principal part of a coil of an insertion portion in a guide sheath according to a second modification of the first embodiment of the disclosure. In the second modification, a guide for guiding insertion of the operating rod 30 into the coil 11 is arranged to the configuration of the first embodiment described above. The configuration other than the guide is the same as that of the first embodiment. Hereinafter, a configuration (guide 14) different from the first embodiment described above will be explained.

The guide 14 is formed in a rectangular shape in which the operating rod 30 can slide therein, matching with the sectional shape of the operating rod 30, and is arranged inside the coil 11. The guide 14 guides an insertion direction of the operating rod 30 as the operating rod 30 is inserted therein. To the coil 11, a plurality of guides 14 are provided. In the third modification, the respective guides 14 are attached to an inner portion of the coil 11 every predetermined number of winding turns. The guides 14 are joined to the coil 11 by laser welding, brazing or solder, adhesion, resistance welding, or the like.

According to the second modification explained above, effects similar to those of the first embodiment described above can be obtained, and by guiding the operating rod 30 by the guides 14, it is possible to suppress deviation of an insertion position of the operating rod 30 with respect to the coil 11, and to suppress buckling thereof. By increasing the number of the guides 14, arrangement intervals of the guides 14 can be made small (narrow), and thereby suppresses buckling of the operating rod 30.

Third Modification of First Embodiment

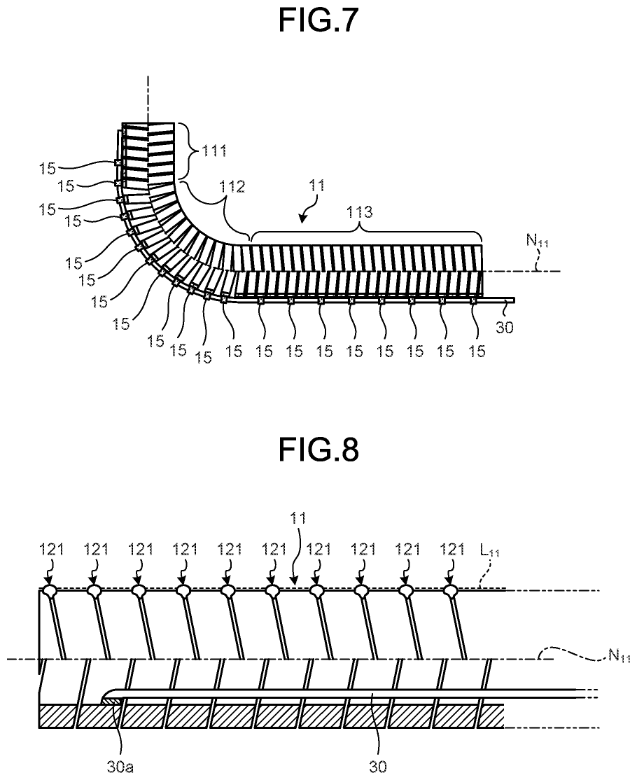

FIG. 7 is a cross-section for explaining a configuration of a principal part of a coil of an insertion portion in a guide sheath according to a third modification of the first embodiment of the disclosure. In the third modification, a guide to guide insertion of the operating rod 30 to the coil 11 is provided to the configuration of the first embodiment. The configuration other than the guide is the same as that of the first embodiment. Hereinafter, the configuration (guide 15) different from the first embodiment described above will be explained.

The guide 15 is formed in a rectangular shape in which the operating rod 30 can slide therein, matching with the sectional shape of the operating rod 30, and is arranged outside the coil 11. The guide 15 guides an insertion direction of the operating rod 30 as the operating rod 30 is inserted therein. To the coil 11, a plurality of guides 15 are provided. In the fourth modification, the respective guides 15 are attached to an outer portion of the coil 11 every predetermined number of winding turns. The guides 15 are joined to the coil 11 by laser welding, brazing or solder, adhesion, resistance welding, or the like.

According to the third modification explained above, effects similar to those of the first embodiment described above can be obtained, and by guiding the operating rod 30 by the guides 15, it is possible to suppress deviation of an insertion position of the operating rod 30 with respect to the coil 11, and to suppress buckling thereof.

Although examples in which separate guides are provided to the coil 11 have been explained in the second and the third modifications described above, a structure integrated with the coil 11, for example, a structure in which a part of the coil 11 is formed in a U-shape, and the operating rod 30 is inserted through a hole that appears as a result of this formation of the U-shape, may be adopted.

### Second Embodiment

FIG. 8 is a partial cross-section for explaining a configuration of a principal part of a coil of an insertion portion in a guide sheath according to a second embodiment of the disclosure. The second embodiment differs from the first embodiment described above from a point in which parts of a coil are joined to each other. A configuration other than this point is the same as that of the first embodiment. Hereinafter, the point different from the first embodiment described above will be explained.

The coil 11 according to the second embodiment is formed in such a manner that adjacent parts of the wire in the direction of the spiral axis N₁₁ are joined to each other. Specifically, in the coil 11, plural elastic joint portions 121 that are aligned in the direction of the spiral axis N₁₁ are formed. Each elastic joint portion 121 is formed with an elastic body joining adjacent parts of the wire to each other in the direction of the spiral axis N₁₁. The elastic body is formed by, for example, laser welding. The respective elastic joint portions 121 are arranged along a generating line L₁₁. The elastic body may be formed by brazing or solder, adhesion, resistance welding, or the like as well as the laser welding, to join the adjacent parts of the wire to each other. The adjacent parts of the wire of the coil 11 are bridged with adherend of melted themselves, or adherend of brazing, solder, adhesive, or the like. The elastic joint portion 121 forms a bendable adjusting unit.

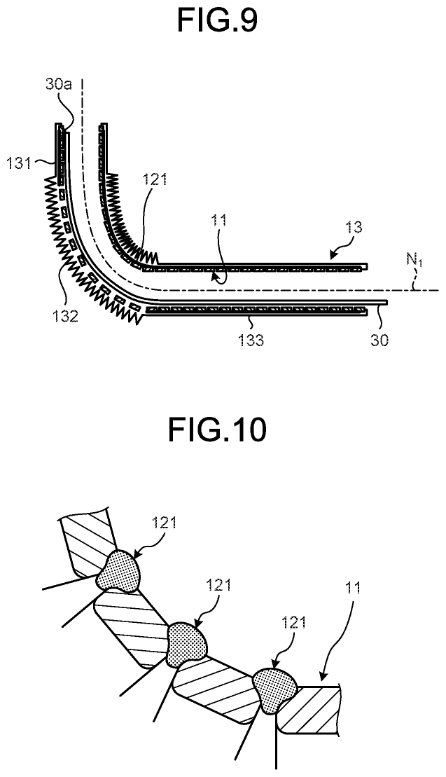

FIG. 9 is a cross-section for explaining the insertion portion 10 in a state in which the bendable portion 102 is bent. FIG. 10 is a cross-section in which a part of the coil illustrated in FIG. 9 is enlarged. FIG. 9 illustrates the coil 11 of the insertion portion in a state in which the bendable portion 102 is bent. Hereinafter, the elastic joint portion can be illustrated omitting the respective elastic bodies, but illustrating its formation area as a representative thereof. When the operating rod 30 is pushed toward the insertion portion 10 (the distal end side) by movement of the operating lever 22, the coil 11 is pulled by the first rigid coil portion 111 and is pressed to be expanded. At this time, the adjacent parts of the wire are fixed to each other with the elastic joint portion 121 in the coil 11, and thus this portion prevents from separating between the adjacent parts of the wire. In the coil 11, a space between the adjacent parts of the wire in which the elastic joint portion 121 is not formed widens to deform the coil 11. The coil 11 is bent as a space between the adjacent parts of the wire on a side in which the operating rod 30 is inserted widens. Moreover, as the bendable coil portion 112 is bent, the stretching portion 132 stretches.

Moreover, a load on the operating rod 30 in pushing can be adjusted by changing the thickness or the width, and a quality of material (elasticity, and the like) of the operating rod 30 in a belt shape extending with a rectangular section maintained.

In the second embodiment explained above, by joining the adjacent parts of the wire of the coil 11 to each other, the bent direction of the bendable portion 102 is controlled. Moreover, because the second embodiment enables to form a bent state with the coil 11 that is constituted of a single member formed by winding the wire, unlike a configuration in which an insertion portion is bent by plural angle rings, it is possible to assemble a bendable tube (coil 11 in this example) easily, and to reduce time and effort of an assembly work because the bendable tube can be handled as a unit. Moreover, according to the second embodiment, because a bending operation is enabled by the operating rod 30 in a belt shape extending with the rectangular section maintained, it is possible to suppress deformation of the operating rod 30 in a width direction, and to suppress shaking in a vertical direction relative to a direction of bent.

Although an example in which the elastic bodies constituting the elastic joint portions 121 are formed in series in the direction of the spiral axis N₁₁ has been explained in the second embodiment described above, it is not limited thereto and, for example, the elastic bodies may be formed in series in a direction inclined with respect to the spiral axis N₁₁, or may be formed to extend spirally on the outer circumference of the coil 11.

### Third Embodiment

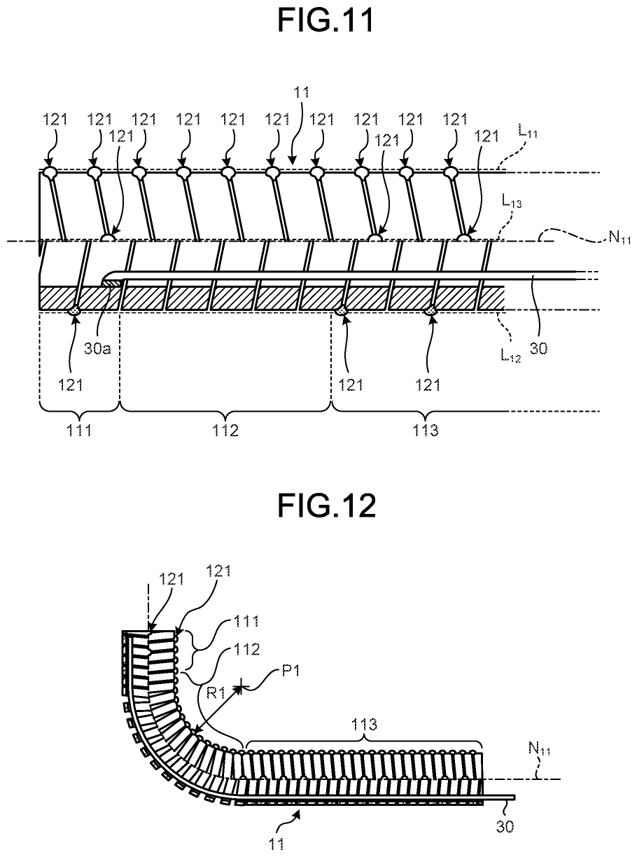

FIG. 11 is a partial cross-section for explaining a configuration of a principal part of a coil of an insertion portion in a guide sheath according to a third embodiment. The third embodiment differs from the second embodiment described above in a point in which an elastic joint portion formed in a coil is added. A configuration other than this point is the same as that of the second embodiment. Hereinafter, the configuration different from the second embodiment described above will be explained.

The coil 11 according to the third embodiment is formed in such a manner that adjacent parts of the wire in the direction of spiral axis N₁₁ are joined to each other. Specifically, in the coil 11, the plural elastic joint portions 121 that are aligned in the direction of the spiral axis N₁₁ are formed. The elastic joint portions 121 are arranged at varied intervals along the spiral axis N₁₁. Each elastic joint portion 121 is formed with an elastic body joining adjacent parts of the wire to each other in the direction of the spiral axis N₁₁. In an area corresponding to the first rigid coil portion 111 and an area corresponding to the second rigid coil portion 113, the elastic joint portions 121 are formed on the generating line L₁₁ to a generating line L₁₄ different from each other (the generating line L₁₄ is not illustrated). Moreover, in an area corresponding to the bendable coil portion 112, the elastic joint portion 121 is formed on the generating line L₁₁. The generating line L₁₂ is across the spiral axis N₁₁ from the generating line L₁₁. The generating lines L₁₃, L₁₄ are generating lines formed at different positions from each other on a second plane perpendicular to a first plane passing through the generating line L₁₁ and the generating line L₁₂.

In the bendable coil portion 112, the elastic joint portion 121 is formed at one position per one winding turn of a wire of the coil 11. On the other hand, in each of the first rigid coil portion 111 and the second rigid coil portion 113, the elastic joint portions 121 are formed at two or more positions per one winding turn of a wire of the coil 11. Because the elastic joint portions 121 are formed at multiple positions per one winding turn in each of the first rigid coil portion 111 and the second rigid coil portion 113, a separation between adjacent parts of the wire of the coil 11 is constrained. The one winding turn of the wire herein corresponds to one pitch of winding the wire about the spiral axis N₁₁.

FIG. 12 is a cross-section for explaining about the insertion portion 10 in a state in which the bendable portion 102 is bent. When the operating rod 30 is pushed toward the insertion portion 10 (the distal end side) by movement of the operating lever 22, the coil 11 is pulled by the first rigid coil portion 111 and is pressed to be expanded. At this time, the adjacent parts of the wire are fixed to each other with the elastic joint portion 121 in the coil 11, and thus this portion prevents from separating between the adjacent parts of the wire. In the coil 11, a space between the adjacent parts of the wire in which the elastic joint portion 121 is not formed widens to deform the coil 11. Specifically, the coil 11 is bent as a space between the adjacent parts of the wire on a side on which the operating rod 30 is inserted (opposite side to the generating line L₁₁) widens in the area corresponding to the bendable coil portion 112. At this time, the bendable coil portion 112 is bent in a state in which the adjacent parts of the wire are connected to each other by the elastic joint portions 121 (for example, refer to FIG. 10). Moreover, as the bendable coil portion 112 bends, the stretching portion 132 stretches.

At this time, the bendable portion 102 (the bendable coil portion 112) is bent according to a pushing amount of the operating rod 30. For example, when the coil 11 is bent in an arc shape with a curvature radius R1 about a point P1, by changing the pushing amount of the operating rod 30, a bent mode of the bendable portion 102 can be adjusted. Specifically, when the operating rod 30 is pushed in more, the curvature radius R1 becomes smaller, and when the pushing amount is reduced, the curvature radius becomes larger.

The bent mode of the bendable portion 102 can be adjusted by changing a range in which the elastic joint portion 121 is formed in the coil 11, thickness of the wire (the thickness or the width if the section is in a rectangular shape), and a quality of material (elasticity, and the like).

Furthermore, a load on the operating rod 30 in pushing can be adjusted by changing the thickness or the width, and a quality of material (elasticity, and the like) of the operating rod 30 in a belt shape extending with a rectangular section maintained.

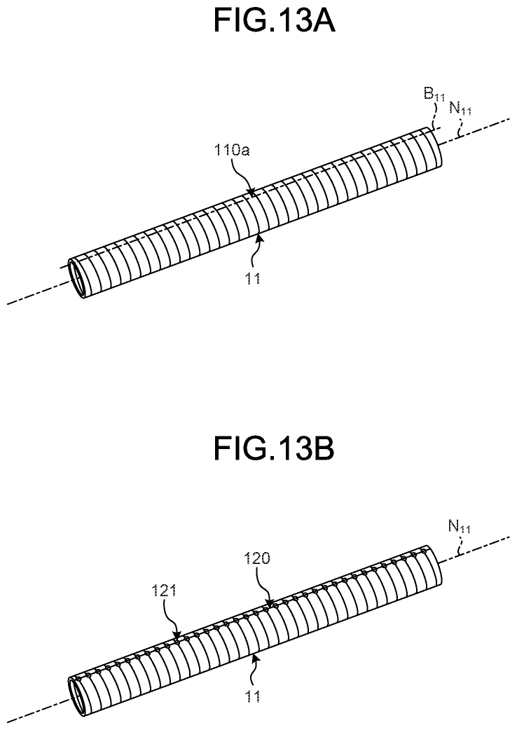

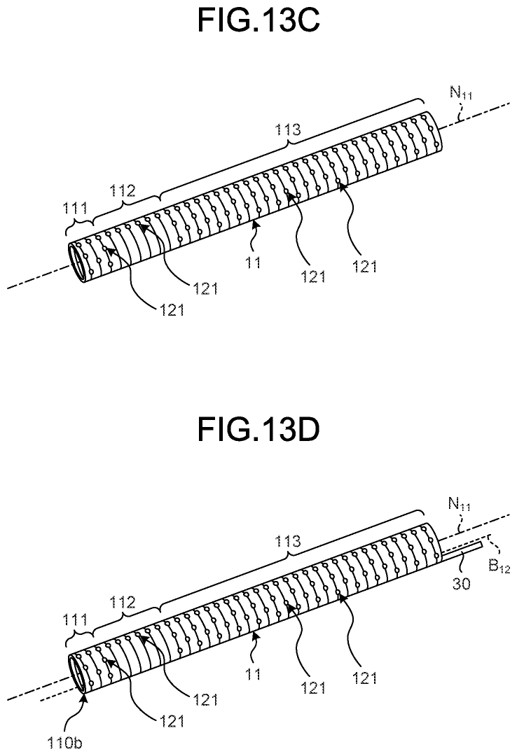

Next, a manufacturing method of the coil 11 having the elastic joint portion 121 described above will be explained with reference to FIG. 13A to FIG. 13D. FIG. 13A to FIG. 13D are diagrams explaining the manufacturing method of the coil in the guide sheath according to the third embodiment of the disclosure. First, a single piece of a junction line 120 is formed from one end to the other end in the direction of the spiral axis N₁₁ by laser welding (refer to FIG. 13B) for the tightly wound coil 11 (refer to FIG. 13A). The junction line 120 is formed by intermittently irradiating a laser beam while moving an irradiation position of the laser beam along the spiral axis N₁₁ to successively form plural elastic joint portions (elastic bodies). The junction line 120 is formed on a first side portion 110a that is arranged at one portion (a part) in a circumferential direction of the coil 11, and on the first side portion 110a along a generating line B₁₁ in the direction of the spiral axis N₁₁. This generating line B₁₁ corresponds to the generating line L₁₁ described above.

The laser beam is emitted by using a device that is capable of controlling oscillation cycles in a nanosecond to several second rate. Moreover, the laser beam is preferable to be generated by using a device capable of controlling an irradiation range, such as a fiber laser, from the viewpoint of controlling a welding position and a welding range.

By forming the junction line 120, elastic bodies constituting a part of the junction line 120 are formed, and thereafter, plural elastic joint portions 121 are formed according to arrangement position of the first rigid coil portion 111 and the second rigid coil portion 113 (refer to FIG. 13C). The respective elastic joint portions 121 are formed by irradiating a laser beam intermittently while parallelly moving an irradiation position of the laser beam relative to the spiral axis N₁₁. At this time, at a position at which the irradiation position of the laser beam corresponds to an area in which the bendable coil portion 112 is formed, the laser beam is not irradiated. For example, by irradiating the laser beam while rotating the laser emitting position or rotating the coil at a predetermined angle (for example, 90°), the respective elastic joint portions are formed. When the joint portions are formed while rotating at 90°, a structure in which four pieces of elastic bodies are aligned in the circumferential direction per single winding turn of the wire is obtained. Other than that, when the joint portions are formed while rotating at 60°, a structure in which six pieces of the joint portions are aligned in the circumferential direction is obtained. In the first rigid coil portion 111 and the second rigid coil portion 113, a rigid coil portion constraining a separation between the adjacent parts of the wire by the elastic joint portions 121 is constructed.

After forming the elastic joint portion 121, the operating rod 30 is inserted in the coil 11, and the distal end of the operating rod 30 is fixed inside (front end portion) the first rigid coil portion 111 (refer to FIG. 13D). At this time, the operating rod 30 is arranged along a second side portion 110b across the spiral axis N₁₁ from the first side portion 110a. The second side portion 110b is arranged in parallel with the spiral axis N₁₁, and along a generating line B₁₂ across the spiral axis N₁₁ from the generating line B₁₁. That is, the operating rod 30 extends along the second side portion 110b in the coil 11. This generating line B₁₂ corresponds to the generating line L₁₂ described above.

After arranging the operating rod 30 in the coil 11, the operating portion 20 is attached to the coil 11, and the operating rod 30 is fixed to the operating lever 22. Thereafter, by covering an outer periphery of the coil 11 with the cover member 13, the guide sheath 1 is manufactured.

In the third embodiment described above, by adjusting a joint position of the wire of the coil 11 (position at which the elastic joint portion 121 is formed), a bent direction of the bendable portion 102 is controlled. According to the third embodiment, because it is possible to make a bent state with the coil 11 that is constituted of a single member formed by winding the wire, unlike a configuration in which an insertion portion is bent by plural angle rings, it is possible to assemble a bendable tube (coil 11 in this example) easily, and to reduce time and effort of an assembly work because the bendable tube can be handled as a unit. Moreover, according to the third embodiment, because a bending operation is enabled by the operating rod 30 in a belt shape extending with the rectangular section maintained, it is possible to suppress deformation of the operating rod 30 in a width direction (direction perpendicular to a plane passing through the longitudinal axis N₁, the first side portion 110a and the second side portion 110b in this example), and to suppress shaking in a vertical direction relative to a direction of bent.

Furthermore, according to the third embodiment, because a bendable mechanism is formed by using the coil 11 constituted of wound the wire, it is possible to reduce the number of parts and to simplify the structure compared to the configuration of bending the insertion portion by plural angle rings, and to make assembly of the insertion portion easy.

Although an example in which plural welding portions constituting the joint portions are formed in series in the direction of the spiral axis N₁₁ has been explained in the third embodiment, it is not limited thereto and, for example, the welding portions may be formed in series in a direction inclined with respect to the spiral axis N₁₁, or may be formed to extend spirally on the outer circumference of the coil 11.

First Modification of Third Embodiment

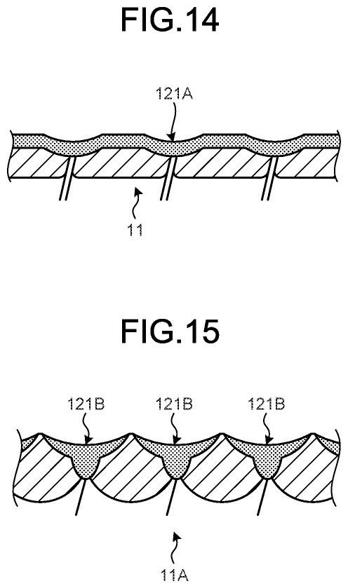

FIG. 14 is a cross-section for explaining a configuration of a principal part of an insertion portion in a guide sheath according to a first modification of the third embodiment of the disclosure. The first modification differs from the third embodiment described above in a joining form of a joint portion. A configuration other than the joint portion is the same as that of the third embodiment. Hereinafter, the configuration (elastic joint portion) that differs from the third embodiment described above will be explained.

In the coil 11 according to the first modification, an elastic joint portion 121A is formed. The elastic joint portion 121A is constituted of a continuous elastic body that is, for example, formed by melting and solidifying the wire by a laser beam. The elastic joint portion 121A extends in the direction of the spiral axis N₁₁ of the coil 11. The elastic joint portion 121A is formed, for example, by repeating spot welding in the direction of the spiral axis N₁₁. Specifically, the elastic joint portion 121A is formed by irradiating a laser beam, overlapping welding areas adjacent to each other in the direction of the spiral axis N₁₁. Brazing, solder, adhesion, resistance welding, and the like instead of the laser welding may be adopted.

According to the first modification explained above, also when the form of joining the adjacent parts of the wire of the coil 11 is made as the elastic joint portion extending in the direction of the spiral axis N₁₁, similar effects as the third embodiment can be obtained.

The joining form of the third embodiment described above and the joining form of the first modification may be combined as appropriate according to a joint portion. For example, the form of forming the elastic joint portions intermittently explained in the third embodiment may be adopted to an area corresponding to the bendable coil portion, and the continuous form made by welding explained in the first modification may be adopted to an area corresponding to the first rigid coil portion and the second rigid coil portion.

Second Modification of Third Embodiment

FIG. 15 is a cross-section for explaining a configuration of an insertion portion in a guide sheath according to a second modification of the third embodiment of the disclosure. The second modification differs from the third embodiment described above in a sectional shape of a wire of a coil. Hereinafter, a component (coil) different from the third embodiment described above will be explained.

A coil 11A is constituted of a wire that extend with a circular sectional plane maintained and that is wound in a spiral form. In the coil 11A, the elastic joint portions 121B to joint adjacent parts of the wire to each other in the direction of the spiral axis N₁₁ is formed, similarly to the first embodiment. The elastic joint portions 121B are constituted of plural elastic bodies, for example, formed by welding and solidifying the adjacent parts of the wire by a laser beam. When a weld portion is formed between the adjacent parts of the wire having a circular section, a radial weld portion of a meltage run into a gap between the adjacent parts of the wire is formed. This elastic joint portions 121B are formed by forming elastic bodies continuously in the direction of the spiral axis N₁₁. The adjacent parts of the wire may be joined to each other by brazing, solder, adhesion, resistance welding, or the like instead of the laser welding.

According to the second modification explained above, also when the sectional shape of the wire constituting the coil is a circular shape, similar effects as the first embodiment described above can be obtained.

### Fourth Embodiment

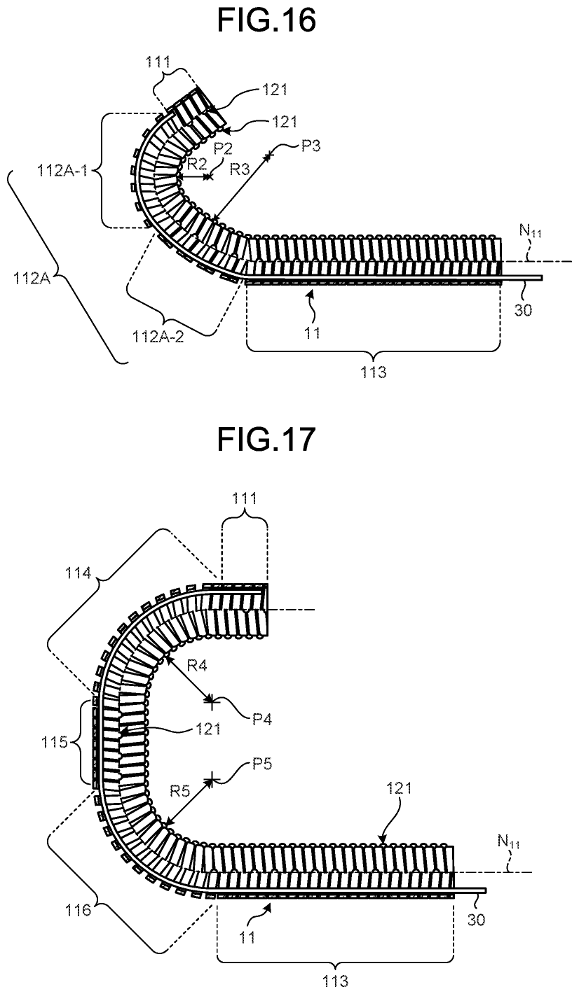

FIG. 16 is a diagram explaining a configuration of a coil of an insertion portion in a guide sheath according to a fourth embodiment of the disclosure. The fourth embodiment differs from the third embodiment described above in a bending form of a bendable portion in the insertion portion. Hereinafter, the configuration different from the third embodiment described above will be explained. FIG. 16 illustrates the coil 11 and the operating rod 30, and shows a state in which the coil 11 is bent.

In the fourth embodiment, the coil 11 has a tightly wound structure in which adjacent parts of the wire in a direction in which it extends as it is wound (corresponding to the spiral axis N₁₁ described above) are in contact with each other. The coil 11 includes the first rigid coil portion 111 extending to correspond to an area in which the distal end portion 101 is formed, the bendable coil portion 112A extending to correspond to the area in which the bendable portion 102 is formed, and the second rigid coil portion 113 extending to correspond to the area in which the proximal end portion 103 is formed.

The bendable coil portion 112A includes a first bendable coil portion 112A-1, a portion of which is joined with the elastic joint portion 121, and that is configured to deform in a predetermined direction, and a second bendable coil portion 112A-2, a portion of which is joined with the elastic joint portion 121, and that is configured to deform at a curvature radium larger than the first bendable coil portion 112A-1.

The elastic joint portions 121 of the respective bendable coil portions are constituted of elastic bodies that join the predetermined umber of adjacent parts of the wire to each other at predetermined positions. The respective elastic joint portions are formed by melting and solidifying the adjacent parts of the wire by a laser beam. Specifically, in an area corresponding to the second bendable coil portion 112A-2, the elastic joint portions 121 are arranged intermittently in the direction of the spiral axis N₁₁ at plural positions per single winding turn. In the fourth embodiment, the elastic joint portion bridges two parts of the wire. The parts of the wire may be joined to each other by brazing, solder, adhesion, resistance welding, or the like instead of the laser welding.

When the operating rod 30 is pushed toward the insertion portion 10 (left side in FIG. 16) by movement of the operating lever 22, the coil 11 is pulled by the first rigid coil portion 111 and is pressed to be expanded. At this time, the adjacent parts of the wire are fixed to each other with the elastic joint portion 121, and thus this portion prevents from separating between the adjacent parts of the wire. In the coil 11, a space between the adjacent parts of the wire in an area in which the elastic joint portion 121 is not formed between the first rigid coil portion 111 and the second rigid coil portion 113 widens to deform the coil 11. Specifically, in the first bendable coil portion 112A-1, a space between the adjacent parts of the wire not joined to each other by the elastic joint portion 121 widens to bend the first bendable coil portion 112A-1. In the second bendable coil portion 112A-2 also, a space between the adjacent parts of the wire not joined to each other by the elastic joint portion 121 widens to bend the second bendable coil portion 112A-2.

For example, when the first bendable coil portion 112A-1 is bent in an arc shape with a curvature radius R2 about a point P2, and the second bendable coil portion 112A-2 is bent in an arc shape with a curvature radius R3 about a point P3 by a push (displacement) of the operating rod 30, because the adjacent parts of the wire of the first bendable coil portion 112A-1 are partially joined to each other by the elastic joint portion 121, the curvature radius R2 becomes smaller than the curvature radius R3.

In the fourth embodiment explained above, by adjusting a joint position of the wire of the coil 11, a bent direction of the bendable portion 102 (refer to FIG. 1) is controlled. According to the fourth embodiment, unlike a configuration in which an insertion portion is bent by plural angle rings, because a bent state can be made with the coil 11 that is constituted of a single member formed by winding the wire, it is possible to assemble a bendable tube easily, and because the bendable tube can be handled as a unit, time and effort of an assembly work can be reduced. Moreover, because a bending operation is enabled by a rod in a belt shape extending with the rectangular section maintained, it is possible to suppress deformation in a direction of the width of the rod, and to suppress shaking in a vertical direction relative to a direction of bent.

Moreover, in the fourth embodiment, because a bendable mechanism is formed by using the coil 11 constituted of wound the wire, it is possible to reduce the number of parts and to simplify the structure compared to the configuration of bending the insertion portion by plural angle rings, and to make assembly of the insertion portion easy.

Furthermore, in the fourth embodiment, a bending form of the bendable coil portion 112A including the first bendable coil portion 112A-1 and the second bendable coil portion 112A-2 in which the adjacent parts of the wire are partially joined to each other is different according to the joining form of the elastic joint portion 121. In the fourth embodiment, by changing a joint portion (for example, the number of the adjacent parts of the wire to be bridged with the elastic joint portion 121 in the second bendable coil portion 112A-2) in a deformation portion, a bent shape of the bendable coil portion 112A can be changed, or a curvature radius can be partially adjusted.

### Fifth Embodiment

FIG. 17 is a diagram for explaining a configuration of a coil of an insertion portion in a guide sheath according to a fifth embodiment of the disclosure. The fifth embodiment differs from the first embodiment described above in a bending form of a bendable portion in an insertion portion. Hereinafter, a different configuration from the first embodiment described above will be explained. FIG. 17 illustrates the coil 11 and the operating rod 30, and shows a state I which the coil 11 is bent.

In the fifth embodiment, the coil 11 has a tightly wound structure in which adjacent parts of the wire in a direction in which it extends as it is wound (the spiral axis N₁₁ described above) are in contact with each other. The coil 11 includes the second rigid coil portion 113 that is arranged on the proximal end side of the insertion portion, the first rigid coil portion 111 that is arranged on the distal end side of the insertion portion, a first bendable coil portion 114 that extends from the first rigid coil portion 111 and that deforms by displacement of the operating rod 30, an intermediate rigid coil portion 115 that extends toward the opposite side to a first rigid coil portion 111 from the first bendable coil portion 114, and a second bendable coil portion 116 that extends toward the opposite side to the first bendable coil portion 114 from the intermediate rigid coil portion 115 to be connected to the second rigid coil portion 113, and that deforms by displacement of the operating rod 30. In the cover member, a portion corresponding to the intermediate rigid coil portion 115 may take a non-stretching form (not in a bellows shape).

Moreover, the coil 11 is formed by joining adjacent parts of the wire to each other in a direction of axis extending as the wire is wound (corresponding to the direction of the spiral axis N₁₁). Specifically, in the coil 11, elastic joint portions 121 described above are formed. Specifically, in each of the first rigid coil portion 111, the second rigid coil portion 113, and the intermediate rigid coil portion 115, plural elastic joint portions 121 are formed per single winding turn, and a separation between the adjacent parts of the wire is constrained. On the other hand, in each of the first bendable coil portion 114 and the second bendable coil portion 116, a single piece of the elastic joint portion 121 is formed along a shared generating line (for example, the generating line L₁₁) per single winding turn of a wire. The elastic joint portions are constituted of plural elastic bodies that are formed by welding and solidifying between the adjacent parts of the wire by a laser beam. The adjacent parts of the wire may be joined to each other by brazing, solder, adhesion, resistance welding, or the like instead of the laser welding.

When the operating rod 30 is pushed toward the insertion portion 10 (left side in FIG. 17) by movement of the operating lever 22, the coil 11 is pulled by the first rigid coil portion 111 and is pushed to be expanded. At this time, the adjacent parts of the wire are fixed to each other with the elastic joint portion 121 in the coil 11 and thus this portion prevents from separating between the adjacent parts of the wire. In the coil 11, a space between the adjacent parts of the wire in an area in which the elastic joint portion 121 is not formed in the first bendable coil portion 114 and the second bendable coil portion 116 widens to deform the coil 11.

By pushing of the operating rod 30, for example, the first bendable coil portion 114 is bent in an arc shape with a curvature radius R4 about a point P4, and the second bendable coil portion 116 is bent in an arc shape with a curvature radius R5 about a point P5. At this time, when the width of the wire (length of the wire in the direction of the spiral axis of the coil in this example) and the number of winding turns are the same, the curvature radius R4 and the curvature radius R5 are to be the same.

In the fifth embodiment explained above, by adjusting a joint position of the wire of the coil 11, a bent direction of the bendable portion 102 (refer to FIG. 1) is controlled. According to the fifth embodiment, unlike a configuration in which an insertion portion is bent by plural angle rings, because a bent state can be made with the coil 11 that is constituted of a single member formed by winding the wire, it is possible to assemble a bendable tube easily, and because the bendable tube can be handled as a unit, time and effort of an assembly work can be reduced. Moreover, because a bending operation is enabled by a rod in a belt shape extending with the rectangular section maintained, it is possible to suppress deformation in a direction of the width of the rod, and to suppress shaking in a vertical direction relative to a direction of bent.

Moreover, in the fifth embodiment, because a bendable mechanism is formed by using the coil 11 constituted of wound the wire, it is possible to reduce the number of parts and to simplify the structure compared to the configuration of bending the insertion portion by plural angle rings, and to make assembly of the insertion portion easy.

Furthermore, in the fifth embodiment, the first bendable coil portion 114 and the second bendable coil portion 116 in which the adjacent parts of the wire are partially joined to each other with the elastic joint portion 121 are aligned in the direction of the spiral axis of the coil 11 with the intermediate rigid coil portion 115 intervened therebetween. In the fifth embodiment, by providing a deforming portion through the intermediate rigid coil portion 115, it is possible to change the bending form, or to partially adjust the curvature radius. Moreover, by providing plural intermediate rigid portions and providing bendable portions at both ends of the intermediate rigid portion, the bendable portion can be bent in multiple stages.

Embodiments to implement the disclosure have so far been explained, the disclosure is not to be limited to the embodiments described above. For example, in the embodiments described above, a configuration of a medical guide sheath to be punctured to a subject has been explained as an example, it can be used as an industrial guide sheath to observe inside by puncturing into a structure, and the like.

In the embodiments and modifications described above, it has been explained that laser welding is performed by a laser beam, but a welding method is not limited thereto. For example, publicly known techniques, such as arc welding, spot welding, and electron beam welding, can be used.

Moreover, in the first to the fifth embodiments described above, an example in which a coil has a tightly wound structure in which the adjacent parts of the wire are in contact with each other has been explained, but a gap may be formed between the adjacent parts of the wire within a range enabling joint by the joining portion.

Furthermore, in the elastic joint portion of the first to the fifth embodiments described above, a configuration in which a weld portion formed by melting the wire itself serves as an elastic body and joins the adjacent parts of the wire to each other has been explained, but as long as a material has elasticity, a material different from the wire, for example, brazing, solder, adhesive, or the like may be used to join the adjacent parts of the wire to each other. Moreover, an example in which an elastic joint portion joins the adjacent parts of the wire to each other in the direction of the spiral axis N₁₁ with a single elastic body has been explained, but a structure in which plural elastic bodies are formed in a single elastic joint portion may be adopted.

Furthermore, in the first to the fifth embodiments described above, an example in which an elastic joint portion is constituted of plural elastic bodies (for example, the elastic body 121a) has been explained, but as long as bent of the coil can be prevented, a structure in which the elastic body is formed at one position can be adopted. At this time, the elastic body is not limited to be one extending parallelly to the spiral axis, but may be one extending in the spiral form.

Furthermore, in the first to the fifth embodiments described above, an example in which a configuration of a coil has a tightly wound structure in which the adjacent parts of the wire in the direction of the spiral axis are in contact with each other has been explained, but a gap may be formed between the adjacent parts of the wire within a range enabling joint by the elastic joint portion.

The disclosure can include various embodiments within a range not departing from a technical ideas described in claims.

As described above, the bendable tube unit of the medical guide sheath according to the disclosure enables a bendable tube to be assembled easily, and is useful for reducing time and effort of an assembly work of the bendable tube.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A bendable tube unit of a medical guide sheath, the bendable tube unit comprising,
a coil in which an elastic wire is wound in a spiral form, and
a rod that is arranged inside the coil along and near a generating line in a circumferential direction of the coil, and that has a distal end portion and a rear end portion, the distal end portion being fixed to a front end portion of the coil, the rear end portion being connected to an operating portion configured to apply a bending action to the coil, the rod being configured to bend the coil by moving toward a distal end side of the coil to apply a stretching force to widen a space between parts of the wire near the generating line deviated from an axis of the coil.

2. The bendable tube unit according to claim 1, further comprising an elastic joint portion that is arranged along and near a generating line in the circumferential direction of the coil, the elastic joint portion being configured to join adjacent parts of the wire to each other in a direction of a spiral axis of the coil with at least one elastic body to partially constrain stretching of the coil and to leave rotatability of the coil.

3. The bendable tube unit of a medical guide sheath according to claim 1, wherein
the rod is one of a plurality of rods along and near generating lines at equal angles in the circumferential direction of the coil about a spiral axis of the coil.

4. The bendable tube unit of a medical guide sheath according to claim 2, wherein
the elastic joint portion is one of a plurality of elastic joint portions that are arranged along and near a generating line of the coil across the spiral axis from the rod.

5. The bendable tube unit of a medical guide sheath according to claim 1, wherein
the rod includes a belt-shaped material extended to have a uniform rectangular cross-section.

6. The bendable tube unit of a medical guide sheath according to claim 2, wherein
the coil includes a rigid coil portion in which a plurality of elastic joint portions are arranged at varied intervals in the direction of the spiral axis and are configured to join the adjacent parts of the wire to each other in the direction of the spiral axis at two or more positions per one winding turn to constrain a separation between the adjacent parts of the wire.
